(19) United States
(12) Patent Application Publication
(10) Pub. No.: US 2006/0263884 A1
(43) Pub. Date: Nov. 23, 2006

(54) Use of passageways through porous membranes

(76) Inventor: Roy H Hammerstedt, (US)

Correspondence Address: THE WEBB LAW FIRM, P.C., 700 KOPPERS BUILDING, 436 SEVENTH AVENUE, PITTSBURGH, PA, 15219, US

(21) Appl. No.: 10/542,822

(22) PCT Filed: Jan. 21, 2004
(86) PCT No.: PCT/US04/01489

§ 371 Date: Apr. 20, 2006

(30) Foreign Application Priority Data
Jan. 21, 2003 (US) 60441396

(51) Int. Cl. G01N 33/00

(52) U.S. Cl. 436/6

(57) ABSTRACT

Capillary-pore (track-etched) membranes were known to have residual negative charges formed during manufacture. We demonstrated that residual negative charges were concentrated on the interior face of the uniform passageways through the membrane stock, and then demonstrated that they were from carboxyl groups (ca 40 nanomoles per cm2 of membrane surface). We then demonstrated that these endogenous carboxyl groups could be used for modification of the surface of these highly uniform passageways, by covalent linkage with one or more compounds, thus providing a configured separation membrane.

### BACKGROUND OF THE INVENTION

A large number of plastics and other materials have been used to make membranes. Among other schemes, membranes might be characterized by general structure (regardless of membrane stock), uniformity of transmembrane passageways, localization of surface properties, and use.

Based on general structure resulting from manufacture, there are: (a) fibril membranes with a “haystack” structure, where limits to a separation are accomplished by tortuous passageways of varying size through a criss-crossed bed of filter materials; (b) microporous membranes with a “sponge-like” structure, where tortuous pores of varying size are created, with proprietary procedures possibly involving gas extrusion and other mechanisms, during formation of the base membrane material (FIG. 1); and (c) capillary-pore or track-etched membranes with a “tunnel” structure, created in a solid sheet of base membrane material by inducing highly controlled physical damage in the membrane and then etching the damaged areas to create pores or transmembrane passageways of uniform diameter through the membrane (FIG. 2).

Capillary-pore membranes have far greater uniformity than microporous or fibril membranes. For example, literature from one vendor (Millipore Inc) provides illustrations and notes that their “10 micrometer pore” microporous membrane product has approximately 68% passageways of nominal 10 micrometer diameter (range not known), but approximately 32% passageways substantially smaller than the nominal 10 micrometers. In contrast, one vendor (Oxyphen AG) provides illustrations and states that the tunnel-like pores in their capillary-pore membranes are of uniform diameter and all are within ±10% of the stated diameter.

Considering surface properties, it is common to increase value of any membrane filter, after formation of the pores, by one of several chemical modifications of the entire surface. One example is plasma treatment in the presence of ammonia gas to create amine functions on exposed surfaces (Ito et al., 1997) or by activation followed by a plunge into acrylic acid (Ito et al., 1990). Such treatments change transport properties across/through the membrane. Other examples of providing membranes responsive to environmental changes include preparation of: (a) co-polypeptide membranes (Maeda et al., 1984; Kinishita et al., 1994), leading to a responsive base membrane with microdomains of polyamino acids; and (b) asymmetric polypeptide membranes (Kinoshita, et al., 1983), with a two-layered membrane providing a responsive network.

Use of membrane filters to separate molecules and particles by size is commonplace; passage of materials larger than the nominal pore size is limited. For many membranes, such filtration is the only application, and control of the process typically is achieved by limiting access of the solution/suspension to be separated to the membrane surface or by changing transmembrane pressure differential (e.g., via a hand-operated or solenoid valve). An alternative approach to control passage of particles smaller than most pores in a membrane is occlusion of the pores with an erodible material to provide a separation barrier with plugged pores (U.S. Pat. Nos. 5,026,342 and 5,261,870). With such a separation membrane, transmembrane passage is allowed only after appropriate environmental conditions have caused or allowed erosion of the material plugging the pores. Mechanical control devices are unnecessary. Such membranes are useful to initiate controlled passage of materials of appropriate size through the pores of a membrane, but their pores can not be re-closed to slow or stop transmembrane passage of materials in response to further change in environment on either or both faces of the membrane.

Inherent to manufacture of capillary-pore membranes is extensive introduction of charges on the membrane. This is because capillary-pore membranes are produced (Luck, 1983) by physically damaging polymer film in a controlled manner with a beam of heavy ions (e.g., krypton) in a cyclotron. The ions follow a linear path where interaction with polymer chains forming the membrane releases energy to damage molecules in the polymer matrix. Damage represents latent pores, which subsequently are opened by chemical etching (e.g., cycles of alkaline and acid treatment). The application describes the nature and location of these negative charges and assigns previously unrecognized useful and novel value and applications to what were shown to be carboxyl groups formed during membrane manufacture.

Literature on production and use of capillary-pore membranes reveals the following six important points: (a) the general concept of etching thin sheets of solids (Price and Walker, 1962; Fleischer and Price, 1963a, 1963b; Fleischer et al., 1964); (b) the concept of changing properties of the pores by coating them with fatty acid monolayers or non-specific adsorption of protein (Petzny and Quinn, 1969; Quinn et al., 1972; Anderson, 1971); (c) the observations that certain polymers can be very sensitive to environmental conditions, and that quality of images in cellulose acetate butyrate, cellulose acetate and nitrocellulose films depends on how the material was prepared (Kesting, 1971); (d) the observation that track registration at etching requires the presence of oxygen (Kesting, 1971), unstated in previous materials is that this requirement for oxygen presumably allows formation of carboxyl groups; (e) the concept that the tunnels in capillary-pore membranes can serve as sites for attachment of chemically active or ionic groups, and that such membranes have the advantage of a providing a very large inner surface for such attachments (Riedel and Spohr, 1980); (f) a number of reports describe multi-step processes for providing points of attachment to the pore interior (Hicke et al., 1999), that involve chemical or physical modification of all the membrane surfaces to generate a functional group as point of attachment, followed by covalent attachment of the molecule of interest. While useful, the requirement for a multi-step process, often requiring use of solvents etc. that could alter pore features, detracts from the utility of that approach. Although we capitalize on point “e”, we differ from “b” and “f” in that surface modifications are not required before attachment of ligands etc. Rather, the novel approach disclosed herein relies on attachment of active molecules using endogenous carboxyl groups residual from initial manufacture.

Comparison of a spherical support or other membrane approach vs. a capillary-pore membrane support for access to molecules passing through a flow-through device is informative. Effectiveness of any flow-through system for processing or analysis depends on its providing a high potential for interaction of the solute of interest with the surface of the support. Suspensions of spherical beads are common and useful because each individual bead has a high surface area (4πr²) to volume (4/3πr³) ratio (S/V ratio) relative to other geometric constructs. Also, hundreds-to-thousands of such beads can be packed in a column format to allow fluids to percolate through the system to allow any individual solute particle (often <0.005 micrometer) to interact with a surface during transit. Each large radius bead (ca 1000 micrometer) has a S/V ratio of ˜3000, and the assembled system has an advantage that only low hydrostatic pressure (5-50 psi) is needed to force fluid flow past the beads, but the disadvantage that void spaces between beads can allow the small solute molecule to pass through the system without interaction with a bead surface. A smaller radius bead (ca 1 micrometer) has a S/V ratio of ˜3,000,000, and the assembled system has the disadvantage that high hydrostatic pressure (>1000 psi) is needed to force fluid flow past the beads, but the advantage that void spaces between beads are much smaller and a small solute molecule is more likely to interact with a bead during passage through the system. Variants have been described (large microporous beads) to maximize desirable and minimize undesirable features, but all share the requirement for careful mechanical assembly into a device that minimizes potential for channeling (inadvertent formation of a passageway through the assembled bead system to allow a solute molecule to pass without interaction with a bead surface).

Other popular device formats involve tangential flow past parallel coated surfaces, or simple immersion of a coated surface in the solution of interest. Efficiency for the former depends on design of the flow path (i.e., its “twists and turns”) to assure interaction with a wall. Efficiency of the latter depends on mixing after immersion and effective removal of the solid material from the solution.

A capillary-pore membrane system has considerable geometric advantage over the above systems. Calculations made from data of Hicke et al. (1999) and product literature of Oxyphen AG establish that flat sheet membrane (˜25 micrometer thick) of moderate pore diameter (0.4 micrometer) has a S/V ratio of 800,000 within the transmembrane passageways, and the S/A ratio increases many fold when the smallest, commercially-available, pore diameter membranes (0.08 micrometer) are used. Hence, the S/V ratio for the transmembrane passageways in a capillary-pore membrane overlaps or exceeds that of spherical supports. Further, the flat sheet, capillary-pore membrane can be mechanically attached to supporting structures by gluing, ultrasonic welding, etc., to provide a system that does not allow solution to pass without going through a pore. Most importantly, a capillary-pore membrane device requires low hydrostatic pressure (5-50 psi) for flow. To illustrate, a macromolecule of diameter of 0.005 micrometer passing through a capillary-pore of 0.08 micrometer is contained within a pore construct with a diameter of only 13× its size, and passes through a passageway with length of 5000× that of the macromolecule being treated. A properly coated surface within such pore will have a high probability of interacting with that molecule as solute(s) moves though the pore by slow flow or diffusion.

A capillary-pore membrane is optically clear, as manufactured, although they can be bonded to an opaque or colored support. For certain applications, optical clarity and low distortion of a separation device has utility.

Analysis of changes within a solution within a the transmembrane passageways of a capillary-pore membrane is possible, given optical clarity and our discovery that the interior of a transmembrane passageway could be modified. We considered attaching colored or fluorescent materials to interior surface of the transmembrane passageway. If such materials change during interaction with the environment, their optical properties could be examined through use of absorption, fluorescence or other spectroscopic means. It is possible to assemble a series of such membranes, allowing light to pass through (and interact with) the first and then proceed to the next membrane. If each membrane bore a different responsive element, a device could concurrently measure many different molecules or ions. Alternatively, a change in physical property other than optical features could be used.

This application was facilitated by importance of tethered ligands as a general analytical approach. Current goals in design of devices for clinical chemistry (and by extension, biomedical and biotechnological) are to simplify (for the end user), multiplex (to gain information about more than one variable at a time), lower cost (by using disposables and off-the-shelf simple components), and speed return of results to user (allowing more rapid decisions). Transition to “point of care” analyses eliminates need for transfer of a sample to a specialized laboratory, and integrated read out of reaction progress is being integrated into many R&D operations. Fluorescence sensing of analytes is an active area, driven by desire to eliminate radioisotopes while retaining sensitivity (with proper controls, fluorescence can be 100,000 times more sensitive than use of absorbance). The text by Lakowitz (1999) summarized the many assays that have been developed for use in such analyses. Most of these assays depend on use of relatively simple organic compounds possessing a functional group suitable for immobilization to a surface.

An example of a more complex system involved construction of a fusion of the Ca sensitive protein, calmodulin, with two different “green fluorescent proteins” (GFP) to provide a complex where binding of Ca results in adjustment of structure and appearance of a fluorescence resonance energy transfer (FRET) signal (Miyawaki et al . . . ; 1997). The GFP system has the potential to provide unique probes for covalent attachment to the solid substrate. General publications (Baird et al., 1999; Ito et al., 1999; Margolin, 2000; Billinton and Knight, 2001; Chiesa et al., 2001; Griesbeck et al., 2001; Nagai et al., 2002) establish the breadth of approach to custom design of proteins to adjust sensitivity to competing ligands and unique spectral features. Highly sensitive systems to sense calcium were developed (Miyawaki et al., 1997; Nagai et al., 2001). pH sensors exhibiting distinct sensitivities and spectral features have been described (Roby et al., 1998; Elsliger et al., 1999; Kneen et al., 1998; Llopes et al., 1998; Takahaski et al., 2001; Awaji et al., 2001; Nifosi and Tozzini, 2003). Of particular interest are the descriptions of Yi and Schultz (2003) of a genetically engineered, allosteric-based indicator for continuous glucose monitoring by FRET technologies; and of Badugu et al. (2003) for boronate complexes capable of continuous measurement of several carbohydrates.

Transformation of solutes during transmembrane passage through a capillary-pore membrane is possible, as the pores can be configured to provide a “microreactor”. Batch synthetic processes can be inefficient due to product inhibition, and difficulties in separation of substrate, products, and catalysts when they have similar physical features. An alternative approach is to immobilize catalyst within a membrane pore, through which both substrate and product can flow under convective forces. Examples are provided by Hicke et al. (1999), who reported use of a capillary-pore membrane, with follow on studies by (Becker et al., 2002 and Borcherding et al., 2003) with a microporous membrane. In each case, a preliminary treatment of the pores was obligatory to allow attachment of the catalyst.

The studies by Ito et al. (1990, 1992, 1997) revealed several features. (a) In each example and all data provided, flow rate of water through the membrane was changed by no more than ˜30% from the basal rate. Neither control of flow over a substantial range at a pre-determined reduced or increased rate (e.g., between 15 and 90% or 115 and 200% of basal rate) nor complete cessation of flow (i.e., 0% of basal rate) was reported. (b) Reported affects could result from occlusion of only that fraction of pores substantially less than 10 micrometers in diameter (likely >30% of all pores; see above) without altering rate of water flow through the majority of pores (i.e., those actually approximately 10 micrometers in diameter). In this case, pH-driven control of water through most pores would not have been achieved. (c) The mechanism proposed in FIG. 6 of Ito et al. (1997) might be intuitively satisfying, but is unlikely to be a correct and complete description of the phenomenon observed. This is because the polyglutamic acid polymer described, and used, when fully extended would have a length only ˜5% of the approximately 10 micrometer diameter of the passageways that constitute the majority of the pores in the membrane. (d) Measurements and text address only movement of water, and by extension other solvents. There is no consideration of solute movement or, more germane, selective control of transfer of solute or particles through the membrane at diffusion rates ranging between zero and free passage or free and zero passage. Hence, Ito did not teach subject matter disclosed herein. Further, given his data, it is illogical to assume that one skilled in the art would conclude that the teachings of Ito could be used to control fluid flow to the extent disclosed herein.

There are other polymers that might be used to control flow of fluid flow through a capillary-pore membrane. Many examples document the potential for polymers to change conformation in response to alteration of their surrounding environment. For example, certain proteins change shape as a result of irradiation, as evidenced by emission from dye molecules bound from solution to the protein (Lovrien and Linn, 1967; Lovrien et al., 1974). Choice of amino acid composition allows preparation of polyamino acids that respond to changes in solvent conditions (Hughes-Despointes et al., 1993; Holtzer, 1994; Speck et al., 1995).

There is at least one earlier application of protein-based polymers to produce mechanical devices. Urry (1997) coated substrates with one or more polypeptides capable of conformation change induced by alterations of the local environment, such as temperature, chemical potential, electrochemical potential, pressure, or electromagnetic radiation. As an example of a driving force for mechanical work, such peptides were used to connect two solid forms. One solid form was held stationary and the other moved back and forth as a consequence folding and assembly of the peptide, or unfolding and dis-assembly. It might be argued that this useful application of a mechanical force is an example of a pump. However, extension of applications described or implied by Urry for such protein polymers to include formation of an automatic “gate” or “door”, and specifically to provide both the structure for the gate and also the motivational mechanism to open or close the gate are neither taught by Urry nor obvious to one skilled in the art. However, as disclosed herein, molecules such as described by Urry can be positioned within the transmembrane passageways of a capillary-pore membrane and used to control movement of a solute or particle, within a liquid or gaseous phase, through said membrane.

Addition of a molecule to a solution passing through transmembrane passageways of a capillary-pore membrane can be accomplished by release from a tethered (immobilized) affinity ligand or release from a “host-guest” complex. Use of affinity ligands to perform functions is increasing. Extraction can be achieved via tethered affinity ligand with high affinity for specific solutes, often accomplished through use of antibodies, receptor proteins, or highly evolved systems such as biotin-avidin. Borcherding et al. (2003) describe a photografting process with a microporous membrane to allow immobilization of streptavidin for use in removing biotin materials from solutions passing through the pores. They stressed that care must be taken in the photografting process to minimize alteration of the initial pore characteristics. General methods for attaching ligands are provided in Hermanson (1996).

Release of materials from a “host-guest” complex or “preloaded” site into a solution of low concentration of that material passing through a pore or over a sphere is common. One example, chosen to illustrate both the general principle and how selective release of hydrophobic molecules can be achieved in a hydrophilic system, involves cyclodextrins (CDX). Details on CDX molecules and their uses are provided in the compendium of Szejtli and Osa (1996), general review of Uekama and Otagiri (1987) and a thematic issue of Chemical Reviews (D'Souza and Lipkowitz, 1998). The CDX family of molecules have been known for 100 years, and are created by enzymatic conversion of starch by micro-organisms to form toroidally shaped, rigid cyclic oligosaccharides of α (1,4) D-glucopyranosyl bonds. In overview, CDX molecules can be viewed as having an apolar, electron rich hydrophobic interior, a hydrophilic exterior, and entrances to the cavity with a blend of each property. Common natural variants contain either five (termed α-CDX), six (β-CDX) or seven (γ-CDX) glucosyl groups. β-CDX predominates, is least expensive, and has been most highly studied in commercial context. Hundreds of chemical derivatives have been prepared to alter the hydrophobic and hydrophilic balance of both the interior and exterior of the molecule. Cyclodextrins with surface amino groups suitable for use in attaching to a solid support are available (examples in Rekharsky and Inoue, 2002) and an emerging subfield involves use of tethered cyclodextrins as a “host” (Liu et al., 2001a; 2001b), including development of a system where the “guest” molecule can be released from a photoswitchable host (Mulder et al., 2002). There are many other host-guest systems that can be adapted to the capillary-pore system.

### BRIEF DESCRIPTION OF INVENTION

This invention established that the negative charges on a capillary-pore membrane are primarily, if not entirely, within the transmembrane passageways, and also demonstrated that these negative charges indeed are associated with carboxyl groups useful for further covalent linkage. The focus of this invention, however, is a family of flow-through tasks that can be accomplished using an appropriately configured separation membrane created by covalently reacting the carboxyl groups with at least one added compound. Depending on unique properties of the active molecule(s) used to form a configured separation membrane, the membrane can be used to perform tasks such as: (a) to assess and to describe quantitatively changes in a solution, via an integral optical bench and detection of changes in physical and/or chemical properties of the environmentally sensitive ligands; (b) to transform solutes, via catalysts; (c) reversibly to control transmembrane passage of solutions, via environmentally sensitive polymers termed “reversible gates” that expand and contract as solution conditions within the pore change; (d) selectively to add a solute(s) to a solution, via release from an affinity ligand or host-guest complex; and (e) selectively to remove solutes from a solution, via binding to affinity-ligands.

### BRIEF DESCRIPTION OF THE DRAWINGS

FIG. 1 is a micrograph of a typical microporous membrane, showing the large range in pore sizes typical of such membranes. From Millipore Inc promotional materials.

FIG. 2 is two micrographs of a typical capillary-pore membrane, showing the uniform nature of the pores or transmembrane passageways typical of such membranes in surface and cross-sectional views. From Oxyphen AG promotional materials.

FIG. 3 documents association of Neutral Red dye with different capillary-pore membrane materials, showing increased binding as pore size decreases from 10 to 0.6 micrometers and total interior surface area for all pores in the field of view increases. Because Neutral Red dye has a positive charge, this Fig. demonstrates introduction of a negative charge to the membrane.

FIG. 4 documents diminished association of Neutral Red dye with capillary-pore membrane material (0.6 micrometer pores) after prior coupling of ammonia with the endogenous carboxyl groups for different intervals (1-4 hr) to form amides. This elimination of binding of the positively charged dye demonstrates that modification of the membrane removes the negative charge.

FIG. 5 schematically depicts presents the process used to produce FIG. 4, with endogenous carboxyl groups before and after linkage of ammonia to form amides. Endogenous carboxyl groups could be converted to other derivatives (e.g., alcohols, anhydrides, thiol esters, carboxylic esters, etc.) useful for covalent attachment of active molecules.

FIG. 6 documents one of a series of tests using fluoroscein-dextran-lysine, immobilized via linkage to endogenous carboxyl groups within transmembrane passageways of a capillary-pore membrane, to measure pH of the solution filling the pores. This is one example of a configured separation membrane.

FIG. 7 illustrates the relationship between relative concentrations of free and bound forms of a binding protein, which is a function of molecular concentration and dissociation constant. From Lakowicz (1999).

FIG. 8 presents a schematic, cross-sectional, representation of an active molecule covalently attached within a transmembrane passageways of a capillary-pore membrane, when in the compact (left) and expanded (right) forms. This is an example of one use of another configured separation membrane.

### DETAILED DESCRIPTION OF THE INVENTION

Active molecule—In this disclosure, we use the term “active molecule” in a broad sense to include any molecule, combination of molecules, compound, particle, combination of molecules and particles, or material that can be joined to one or more transmembrane passageways, or pores, of a capillary-pore membrane via endogenous carboxyl groups to form a configured separation membrane. Preferred active molecules are environmentally sensitive, but this property is not obligatory. The term active molecule is not restricted to those presented herein, in cited literature, or known today, but rather in the broadest sense to include any appropriate molecule, compound, particle or material later discovered or made. Although generally the active molecule will be covalently bonded to the endogenous carboxyl, attachment by non-covalent means is also contemplated, including but not limited to electrostatic effects.

Affinity-ligand—In this disclosure, we use the term “affinity-ligand” primarily in respect to a molecule that has sufficiently high affinity and specificity for one or more second molecules to give it utility in binding said second molecule for extraction from a solution or for positioning said second molecule for measurement. However, an affinity-ligand also could be used to release a second molecule, by reducing or eliminating the binding forces.

Capillary—In this disclosure, we use the term “capillary” in a broad sense, to include a passageway or tube having a small and more-or-less regular internal diameter.

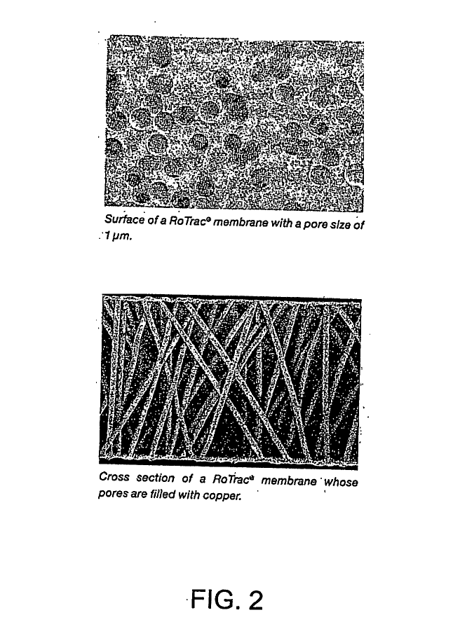

Capillary-pore membrane—In this disclosure, we use the term “capillary-pore membrane” in a broad sense to include any separation barrier with one or more “tunnel-like” passageways extending completely through the barrier, with said tunnel-like passageways created in a solid sheet of base membrane material by inducing highly controlled physical damage in the membrane and then processing the damaged areas of the membrane material to create passageways or pores of uniform, and predictable, diameter through the membrane to form said separation barrier. FIG. 2 shows a typical capillary-pore membrane (without optional backing). Capillary-pore membranes also have been termed, by others, as track-etched or nuclear-track membranes. Although we used RoTrak® capillary-pore membranes for studies as presented, the term and disclosures apply to all capillary-pore membranes.

Carboxyl—In this disclosure, we use the term “carboxyl” to designate the univalent radical, COOH, characteristic of all organic acids. It also is used in the terms carboxyl group (definition obvious) or endogenous carboxyl group (defined below).

Configured separation membrane—In this disclosure, we use the term “configured separation membrane” specifically in respect to a capillary-pore membrane altered by attaching one or more active molecules to one or more transmembrane passageways, through said capillary-pore membrane, by linking said active molecule via endogenous carboxyl groups.

Covalent bond—In this disclosure, we use the term “covalent bond” to designate a chemical bond formed by the sharing of one or more electrons, especially pairs of electrons, between atoms.

Endogenous carboxyl group—In this disclosure, we use the term “endogenous carboxyl group” specifically in respect to one or more COOH groups residual on the face of one or more transmembrane passageways through a capillary-pore membrane as a consequence of manufacture. This specific use of the term endogenous carboxyl group does not preclude location of carboxyl groups on one or both major faces of the membrane, but emphasizes consideration herein on those carboxyl groups actually located within the transmembrane passageways of such a membrane. Hence, claimed properties resulting from active molecules bound to one or more endogenous carboxyl groups occur within the transmembrane passageway or pore and not generally on the major face of the separation barrier. Presence of endogenous carboxyl groups allow formation of a configured separation membrane.

Gate—In this disclosure, we use the term “gate” in respect to an active molecule or polymer which has the capability to change three-dimensional shape, preferably from a compact form to an elongated form or from an elongated form to a compact form, either temporarily or permanently, in response to one or more specific changes in the environment within a transmembrane passageway.

Guest molecule—In this disclosure, we use the term “guest molecule” in respect to a molecule that temporary is attached to or resident within another molecule, termed a “host molecule”.

Host molecule—In this disclosure, we use the term “host molecule” in respect to a molecule that provides a place of temporary attachment or residence for another molecule, termed a “guest molecule”.

Host-guest—In this disclosure, we use the term “host-guest” in respect to a paired set of molecules, where the host molecule provides a place of residence for its partner guest molecule. Displacement of the guest molecule from the host molecule is dependent on features impinging on the equilibrium between “guest paired with host” vs. “guest in surrounding solution”.

Micro—In this disclosure, we use the term “micro” in a general sense, referring to small. Typically, this would refer to a size, diameter, or distance measured in micrometers or fractions of a micrometer, although the context might otherwise define micro.

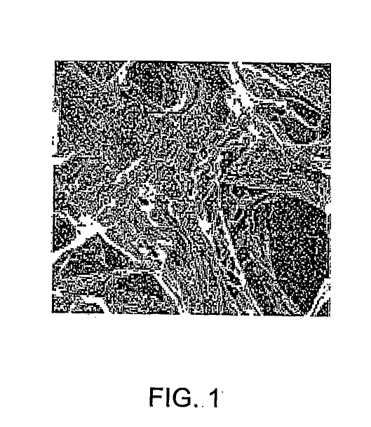

Microporous membrane—In this disclosure, we use the term “microporous membrane” in a general sense in respect to a separation barrier with a “sponge-like” structure, where tortuous pores of varying size are created, with proprietary procedures possibly involving gas extrusion and other mechanisms, during formation of the base membrane material. FIG. 1 shows a typical microporous membrane. The tortuous pores provide one or more passageways through said separation barrier.

Optical-bench—In this disclosure, we use the term “optical-bench” in a general sense in respect uses of a configured separation membrane where an active molecule or a reporter molecule changes in some physical sense, in response to a change of environment within the transmembrane passageways, and this change is detectable by positioning an appropriate detection device in juxtaposition to the surface of the configured separation membrane.

Pore—In this disclosure, we use the term “pore” in a general sense in respect to a micro opening on one surface of a separation barrier and continuous through the thickness of the separation barrier to a micro opening on the opposite face. Hence, a pore serves to provide a passageway through the separation barrier. Many types of separation barriers include pores. Although excluded by our definition of pore, certain separation membranes have “blind pores” that do not provide a transmembrane passageway.

Porous—In this disclosure, we use the term “porous” in a general sense in respect to a separation barrier having a number of pores therein and, hence, allowing transmembrane movement of solvent plus any solutes or ions therein.

Reporter molecule—In this disclosure, we use the term “reporter molecule” in respect to any active molecule pre-positioned during configuration of capillary-pore membrane, when said molecule has properties that allow a change in physical characteristics which can be measured with a detector in juxtaposition to the membrane face. The term also is used in respect to any ion or molecule in a solution later entering a transmembrane passageway after configuration, when said molecule has properties that allow a change in physical characteristics which can be measured with a detector in juxtaposition to the membrane face. Reporter molecules can be used in a cascade reaction where a second reporter molecule enhances the signal from the first reporter.

Reversible gate—In this disclosure, we use the term “reversible gate” in respect to an active molecule or polymer which has the capability to change three-dimensional shape back-and-forth, preferably changing back-and-forth from a compact form to an elongated form, either temporarily or permanently, in response to one or more specific changes in the environment within a transmembrane passageway.

Target molecule—In this disclosure, we use the term “target molecule” in respect to any molecule or ion in a solution entering a transmembrane passageway, during use of a configured separation membrane, where it is bound by an affinity-ligand or host and, hence, removed from the solution.

Task(s)—In this disclosure we use the term “task” or “tasks” in a general sense to refer to any practical use relying on interaction of a solution, ion, or solute with an active molecule contributing to a configured separation membrane.

Transmembrane passageway—In this disclosure, we use the term “transmembrane passageway” in a specific sense in respect to a non-branched pore, of more-or-less uniform diameter, characteristic of a capillary-pore membrane, and a direct result of the manufacturing process of such membranes. Transmembrane passageways, as defined herein, are found only in capillary-pore membranes. The term pore is used interchangeably with transmembrane passage.

The present invention consists of methods which transform a capillary-pore membrane, as supplied by a manufacturer, from a simple separation membrane to a configured separation membrane. Said configured separation membrane is created by covalently linking at least one compound to an endogenous carboxyl group in the capillary-pore membrane. Ordinarily, the configured separation membrane contains one or more active molecules positioned, by covalent linkage with endogenous carboxyl groups, within one or more transmembrane passageways and/or in certain instances on the major face(s) of the membrane. Given the diversity of available, or yet unknown, active molecules, a configured separation membrane could have many uses, some yet unknown or unanticipated. The inventive uses of a configured separation membrane are not restricted to examples provided herein, but rather apply to any similar use of an active molecule positioned within the transmembrane passageway of a capillary-pore membrane.

The present invention is unique because there is no prior art anticipating that any carboxyl group that might be located within the transmembrane passageways of a capillary-pore membrane could be used for a rewarding purpose, e.g., attaching an active molecule to form a configured separation membrane. Further, there is no prior art concerning use of a configured separation membrane, as defined herein, for performance of a “task” by passing a solution of interest through a configured separation membrane at low pressure (e.g., <100 psi). Finally, there apparently is no prior art exploiting the combined benefits of optical clarity of a capillary-pore membrane and positioning of colored or fluorescent active molecules within the transmembrane passageways, via attachment to endogenous carboxyl groups during configuration of a separation membrane, to perform tasks of measurement. This use would be difficult if not impossible with a fibril or microporous membrane, because they typically are opaque and have a highly variable optical path from interior surfaces.

As detailed in examples presented below, a configured separation membrane can be used to: (a) assess and quantitatively describe changes in a solution, via changes in physical and/or chemical properties of the environmentally sensitive ligands; (b) transform solutes, via catalysts; (c) reversibly control transmembrane passage of solutions, via environmentally sensitive polymers (“reversible gates”) that expand and contract as solution conditions within the pore change; (d) selectively add a solute(s) to a solution, via release from ligand traps; and (e) selectively remove solutes from a solution, via affinity ligands.

A configured separation membrane can be used to perform a task either as a flat membrane or after incorporation into a three dimensional construct. The physical geometry of any device can enhance utility of use of a configured separation membrane, but does not affect function of the configured separation membrane except via possibly affecting transmembrane pressure differential, and hence residence time of solute within a given transmembrane passageway and time for interaction with active molecules therein. Claims are not presented for different configurations of devices incorporating a configured separation membrane, or “stack” of configured separation membranes, since logical configurations for constructs are well known or could be deduced by one skilled in the art. For the same reasons, claims in respect to altering or maintaining transmembrane pressure differential are not presented. However, this paragraph is intended to preclude others from filing such device claims.

This invention is restricted to application with capillary-pore membranes, because of unique (compared to fibrillar or microporous membranes) inherent properties including: (a) endogenous (created during manufacture) carboxyl groups on the surface of the membrane; (b) highly uniform pore diameter which, although designed for precise sieving, facilitates reproducible presentation of molecules or ions to active molecules positioned (as disclosed herein) within these pores or transmembrane passageways; (c) reasonably devoid of structural irregularities within a given transmembrane passageway, (d) low resistance to fluid flow allows use with modest transmembrane pressure; and (e) optically clear, yet can be provided with a large variety of laminate backings to provide mechanical stability.

To date all uses of capillary-pore membranes have focused on the “sieving properties” of the membrane (separation by size or hydrophobicity) or have employed cumbersome methods to introduce functional groups on their surfaces after manufacture and prior to attaching molecules to the membrane. Also, in some applications cells or matter trapped on the surface of a transparent capillary-pore membrane, by sieving action, are viewed via transmission of light or electron beams (e.g., phase-contrast microscopy).

Most germane in respect to novelty of this invention, is the fact that although it was recognized that capillary pore membranes have residual negative charges, created during manufacture, their possible differential localization outside (on major faces) or inside the transmembrane passageways apparently had not been reported. Further, acknowledgement that these negative charges were associated with carboxyl groups had never before been made. Our research led us to suspect that the transmembrane passageways must contain a substantial number of carboxyl groups. Subsequent studies, as documented below, demonstrated that this supposition was correct.

We took our determination that many carboxyl groups were within transmembrane passageways of a capillary-pore membrane, and combined it with public knowledge (see background) of the large number of transmembrane passageways and their very favorable S/V ratio, to conclude that capillary-pore membranes provided a porous structure with a vast number of preformed carboxyl groups on which to attach active molecules via covalent linkage. Such modification would add novel value to capillary-pore membranes.

To validate our deductions, we first studied localization of the negative charges. We then activated the putative carboxyl groups and reacted them with amines to form amides, to provide evidence that the negative charges indeed were on carboxyl groups.

The present invention encompasses any conversion of the endogenous carboxyl groups to allow covalent attachment of any active molecule. Such conversions embrace, without limitation, direct reaction to form amides, esters, thiol esters and anhydrides. Additional reactions can be accomplished by conversion of the endogenous carboxyl to an aldehyde or alcohol prior to further reaction with an additional compound such as an active molecule.

We performed studies to estimate the number of endogenous carboxyl groups, and concluded that millions of active molecules could be attached within a given transmembrane passageway. This potential to provide a very high concentration of active molecules per square centimeter of membrane would facilitate accomplishment of a diverse variety of tasks, and add value to a generic capillary-pore membrane. For many applications, the interaction of an active molecule and a target molecule or ion could be described by traditional binding kinetics. This invention is not limited by examples presented. The many diverse uses of active molecules, affinity-ligands, host molecules, reporter molecules, and target molecules, plus the optical bench concept, integral to the concept of configuration of a capillary-pore membrane will be within the skill of the art and each one is included in this patent.

We performed a number of studies to demonstrate the diversity of uses for a configured separation membrane. Use of an environmentally-sensitive chromophore as the active molecule provided an “optical bench” for measurement of pH via changes in intensity of fluorescence. The chromophore-optical bench configuration could be performed with hundreds of other molecules, in an analogous manner, to accomplish diverse tasks involving an analytical measurement of a solution. Further, with appropriate active molecules, the optical bench use of a configured separation membrane could include absorption spectroscopy and other modes of physical analysis. This invention includes any “optical-bench” use of a configured separation membrane.

We evaluated the concept of using a “ligand-trap” as the active molecule, to selectively remove a molecule from a solution on the basis of high and specific affinity of active molecule for the molecules to be removed. Although any antibody-antigen pair or combination well known to those skilled in affinity chemistry could be used, we used avidin as the active molecule to be used as a ligand-trap. We validated the approach by removal of biotin-fluoroscein-dextran-lysine from the solution contacting the thus configured separation membrane. This invention includes any “ligand-trap” use of a configured separation membrane.

### EXAMPLE 1

In Example 1, we determined the primary location of residual carboxyl groups on capillary-pore membrane stock, and also estimated the numbers of residual carboxyl groups on the primary face of a membrane and in transmembrane passageways. We concluded that millions of carboxyl groups were present within each transmembrane passageway.

RoTrac® Capillary-pore membranes (Oxyphen AG), with pore diameters of 0.6, 1.0, 3.0, 5.0 or 10.0 micrometers, were used. They were considered to be representative of other capillary-pore membranes. In their most common format, RoTrac® Capillary-pore membranes are a laminate of: (a) a 25 micrometer thick “polyester” film-membrane (containing the pores, and constituting the separation barrier; see FIG. 2); and (b) an open mesh-like polyester backing (to provide mechanical stability but without separation properties). Materials without laminate, and alternative backing materials, also are available, and their presence or absence does not affect this invention except that a laminate facilitates handling of the capillary-pore membrane.

According to the manufacturer, the fractional area of the film-membrane face occupied by pores, or total “hole area” is constant; i.e., as pore diameter decreases number of pores per unit surface area increases. Since the surface area within a pore (2πrh; where r is pore radius and h is membrane thickness) is highly dependent on pore radius, the total surface area of a membrane within the pores plus the remainder of the two major faces must increase substantially as pore diameter decreases.
We used discs with a 1.2 cm diameter (1.13 cm² surface) for this study, cut from stock sheet membrane materials stated to have pores 0.6, 1.0, 3.0, 5.0 or 10.0 micrometers in diameter. For use as a control materials, we also obtained capillary-pore membrane without the backing material, and base membrane material that had not been converted to a capillary-pore membrane by irradiation and etching.

Because carboxyl groups have a negative charge, we sought a conspicuous dye with a positive charge to study localization. Neutral Red (CI 50040; formula weight=288) has a positive charge below pH 6 and exhibits a red/pink color at pH values below its pKa of 6.7. The amount of Neutral Red in solution at pH 6.0 can be quantified by absorbance at 478 nm (linear between 0 and 0.33 OD; r²=0.99). For these tests, solutions of Neutral Red were prepared at concentrations between 0 and 37 μM (37 μM=ca 10 mg/L).

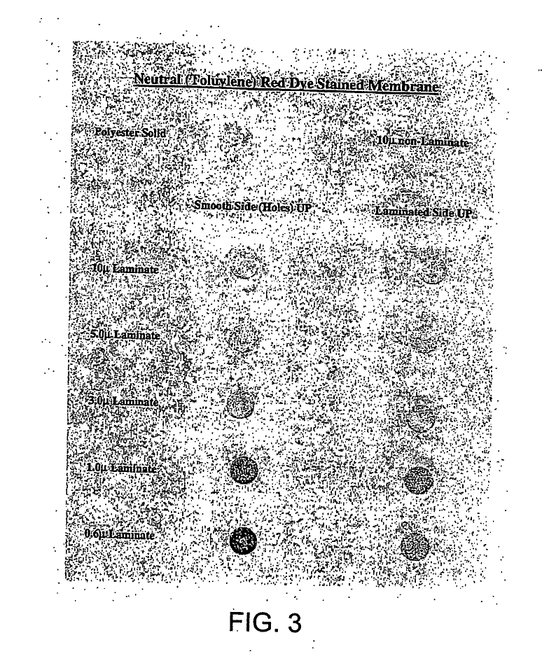

For qualitative observations, we placed discs into a series of vials (1 per vial), covered each disc with 10 mL of 37 μM dye at pH 6, and lightly sonicated each vial for 30-60 sec in a simple laboratory, bath-type sonifier (Branson Inc.) to remove air bubbles from within the pores. After 10 min, the discs were removed, transferred to dye-free buffer at pH 6, treated with light sonication to dislodge loosely bound dye, and air dried. Examination of the filters from one representative study revealed features shown in FIG. 3, in which the two columns show similar membrane discs positioned to show the smooth (active) face (left) or the laminate backing face (right). FIG. 3 shows: (a) Row 1. Left side, no dye was retained by uncharged base membrane material (not been converted to capillary-pore membrane). Right side, dye was associated with discs of capillary-pore membrane (10 micrometer pores) without laminate backing. (b) Rows 2 to 6, discs of membrane ordered by decreasing pore diameter and, hence, increasing surface area within the pores (see above). It was obvious intensity of color increased as size of pore decreased (rows 2 to 6). Further, color was most intense when the smooth (active) face of the laminated membrane was mounted facing the viewer. We concluded that binding of dye was not uniform over the face of a capillary-pore membrane, but rather was associated near and especially within the pores or transmembrane passageways. The data are consistent with our interpretation that this increase in negative charge (from carboxyl groups) on the total surface area of a membrane was a result of the increase in surface area available within transmembrane passageways and that the non-pore portions of the membrane had very few negative charges per unit area.

To study the pKa of the negative charges within the transmembrane passageways, membrane discs with pores 0.6 micrometer in diameter were treated with Neutral Red as above. They then were washed sequentially with buffers at pH 6.0, 5.0, 4.0, 3.0, 2.0 and 1.0. Color was retained after the discs were treated in the first three buffers (between pH 6.0 and 4.0). Partial removal of dye was noted at pH 3. All dye was removed at pH 2. This observation linked with information on how the membranes are manufactured (see background) and observations in FIG. 3 provides strong support for a conclusion that the negative charge in the transmembrane passageways almost certainly is from carboxyl groups.

We also made semi-quantitative measurements of dye binding, by the following sequence of steps. (a) Groups of five membrane discs (pore sizes from no pore; 0.08 to 10 micrometer diameter) were immersed in 50 mL distilled water (pH 6.0) and lightly sonicated to move water into all pores, removed, and then surface water was removed by light “pat” on a paper towel. (b) Discs were transferred into 37 μM Neutral Red, subjected to vortex mixing and lightly sonicated to assure that dye solution entered the pores. Samples then were removed and surface water removed as in (a) above. (c) Discs then were immersed in pH 6.0 water, lightly sonicated, and blotted as in (a) above to remove all non-bound dye. Repeat treatment did not remove additional dye. (d) One group of five discs was immersed in 0.5 N HCl and treated by sonication to displace dye from the discs. Discs were removed. Absorbance of the displaced dye within the solution was measured, along with standards, and used to estimate nanomoles COOH per cm² of disc. Recovered discs appeared colorless and repeat treatment with HCl did not remove additional dye. Data are summarized in Table 1. The same trend was evident for each replicate, and it was evident that the greatest amount of Neutral Red had been bound to and released from membranes with the smallest pore diameter. Only a negligible amount of dye was bound to base membrane (no pore; no irradiation and etching). Data in Table 1 probably provide a minimum estimate of the number of endogenous carboxyl groups per cm² of disc, because loss of dye from a membrane during washing was not accounted for.

Further evidence that the negative charges within the pores of a capillary-pore membrane indeed were provided by carboxyl group was provided by a specific chemical reaction. A carboxyl group can be activated with carbodiimide to form a short-lived ester which, in turn, can react with any of a number of compounds to form a substituted acid function (Hermanson, 1996). We elected to use an amine to form an amide. This requires pH˜5 and a high concentration of amine (protonated form) because the reaction path (to amide vs. hydrolysis by water to reform acid) depends on relative concentrations of the reacting molecules. We could have used an alternative reactants to make different derivatives (e.g., thiol to make thiol ester), as is well known to those skilled in the art.

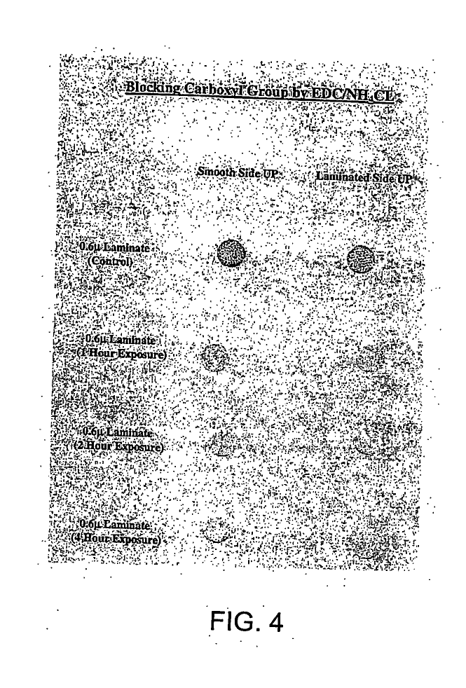

In our studies, discs with 0.6 micrometer pores were treated in the following manner. (a) Discs were suspended in 5.5 M ammonium chloride (pH 4.5) and lightly sonicated to fill the pores. Then 1-ethyl-3-(3-dimethylaminopropyl) carbodiimide (EDC) was added to ˜0.3M, followed by light sonication. The solution was adjusted to pH˜5 with 0.1 N HCl and held at room temperature. After 1, 2 and 3 hr, representative discs were removed from the HCl and washed with water, as described above, and allowed to soak in water (without amine or EDC) overnight, after which they were sonicated lightly, and dried. (b) All discs then were exposed to Neutral Red and examined for retained dye, as described for preparation of FIG. 3. Representative data (FIG. 4) established that few if any negative charges remained after 1 hr, and the contrast with FIG. 3 is obvious. This confirmed our conclusion that negative charges within the transmembrane passageways were from carboxyl groups.

### EXAMPLE 2

In Example 2, we demonstrated that endogenous carboxyl groups could be used for simple and direct attachment of active molecules. Since many molecules of biological interest have an available amine residue, we used histidine, bradykinin, and lysozyme as prototype active molecules to demonstrate attachment to endogenous carboxyl groups, forming a configured separation membrane.

General procedures for a two-step activation of carboxyl groups with subsequent reaction with amines to form stable amides were adapted from among many approaches known to those skilled in the art (Pierce Chemical company; product brochure #24500; Staros et al., 1986).

To activate the carboxyl groups, we added discs of capillary-pore membrane to a mixture of aqueous N-hydroxysuccinimide (10 mM) and N-hydroxysulfosuccinimide (5 mM) in (2-N-Morpholino)ethanesulfonic acid buffer (MES; 0.1 mM; pH 5.0). Vials were sonicated 5-8× for 30 sec each to remove bubbles and assure mixing within pores, and then placed in a 37 C incubator for 20 min. To quench activation reagents, samples then were made 20 mM in 2-mercaptoethanol, held at 37 C for 2 min, and washed 4× with fresh MES buffer (pH 5.0), using sonication to remove excess reagents.

Immediately after activation, each of 5-10 activated membrane discs was added to 20 mL amine (in solution as below), subjected to sonic irradiation for 30 sec, and placed in a 37 C incubator. Each amine of interest (histidine, MW=155; bradykinin, MW=1,060; lysozyme, MW=14,200) was dissolved 10% (v/v) MES buffer (pH 6.0; first using 90% DMSO to solubilize the amine if necessary), to concentrations shown in Table 2. After immersion of a disc in the amine solution, vials were sonicated 6× at 30 min intervals, and then the samples were held overnight 37 C. The next morning, samples were washed 4× in distilled water to assure removal of all unbound amines (verified by control experiments).

Total nitrogen bound to each disc was determined (Janicke, 1974) after perchlorate digestion using ammonium chloride as the standard. Data were expressed as nanomoles amine per cm² of membrane. This is a direct measure of capability of amines of different molecular weight to enter the transmembrane pores and become covalently bound to endogenous carboxyl groups therein. We anticipated a lower degree of incorporation for high molecular weight amines (e.g., lysozyme), than small amines (e.g., histidine) because the size of any previously bound amine could restrict access of additional amines to nearby carboxyl groups.

As shown in Table 2, our assumption that the nanomoles of amine bound was a function of molecular weight proved to be correct. As expected from Example 1, there was a strong tendency for the nanomoles of amine bound to increase as diameter of pores decreased (compare within a row the values for 0.1 vs. 1.0 and especially 0.1 vs. 10 micrometer pores). Note that concentrations of amine tested were not saturating, so that values represent minimal estimates for amounts of tested amines that might be bound.

For histidine, the lowest molecular weight amine tested, and using capillary-pore membranes with pores with 0.1 micrometers diameter, a maximum of 39 nanomoles of amine were attached per cm² of membrane surface (flat face) area. Descriptive material from Oxyphen AG suggests a maximum of 1×10⁹ pores per cm² surface (flat face) area. Hence, we estimated covalent attachment of 4×10⁻¹⁷ moles of amine per pore (or 2.4×10⁺⁷ molecules per pore). We estimated that the internal surface area of a transmembrane passageway (pore) 0.1 micrometer in diameter and 25 micrometers long is 7.9×10⁻¹² m², so that each pore has an amine density of 3×10¹⁸ molecules per m².

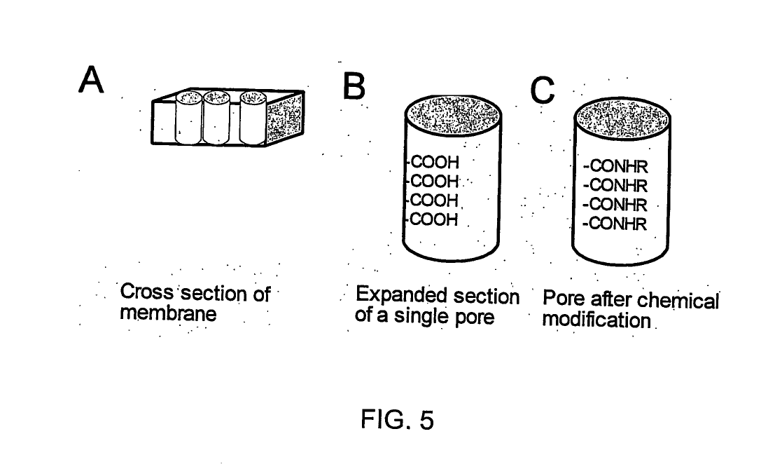

A schematic interpretation of the observations in this example is provided in FIG. 5. Sketch A illustrates the transmembrane passageways of a capillary-pore membrane (as in FIG. 2). Sketch B illustrates the presence of carboxyl groups within a transmembrane passageway, as suggested by data of FIG. 3 and Table 1. Sketch C illustrates a transmembrane passageway after use of the endogenous carboxyl groups to bind an amine, as suggested by data of FIG. 4, to form a configured separation membrane. Carboxyl groups can be converted to many other derivatives (alcohols, anhydrides, thiols, etc.) that could be used to covalently attach other active molecules by methods known to those skilled-in-the-art. This invention includes any conversion of the endogenous carboxyl groups to an active derivative, and linkage of any active molecule thereto.

From data in Examples 1 and 2, we concluded that this large number of carboxyl groups per cm² of capillary-pore membrane, previously unrecognized, are located within the transmembrane passageways and provide a simple mode of attachment of a great variety of active molecules. Such configuration of a capillary-pore membrane, using molecules of known utility via well established chemistries and benign conditions unlikely to modify basic features of the pores, provide many hitherto unknown uses for configured separation membranes. Such uses encompass the spectrum from research, manufacturing, storage, and regulatory situations. A few examples for uses of configured separation membranes are provided below. However, many others will be obvious to those skilled in the arts of biochemistry, biology or medicine; affinity separations or enzymology; treatment and analysis of solutions; covalent attachment of molecules to solid surfaces; and/or fabrication of devices useful for biomedical, agricultural and chemical applications. This invention includes all such approaches for preparation and use of a configured separation membrane.

### EXAMPLE 3

Use of a configured separation membrane as an “optical-bench” for quantitation of changes in a solution, by measurements dependent on light transmission or emission from/by an active molecule, are illustrated in Example 3. Based on Example 2, we estimated that up to 40 nanomoles amines can be covalently bound per cm² to a capillary-pore membrane with pores 0.1 micrometer in diameter. Since the membrane is optically clear, we evaluated use of transmembrane passageways as more-or-less parallel optical benches for spectroscopic analysis. We calculated the amount of flourochrome that might be presented to an fiber-optic probe if the light beam was 0.06 cm in diameter and the probe concurrently interrogated, and received emitted fluorescence from, 3×10⁻³ cm² of membrane. We estimated that up to 0.12 nanomoles of reporter reagent could be monitored, if extent of binding was similar to that obtained with histidine or bradykinin. Note that the active molecule (in this case a “reporter”) is contained within a membrane of only 25 micrometers thick (i.e., light path), and a disc <0.1 cm on diameter might be sufficient. This use of a configured capillary pore-membrane could facilitate design of very small detector compartments (e.g., for intravascular use), with a very high signal for such a small device.

To evaluate fluorescence changes in an appropriately configured separation membrane, we used an Ocean Optics Inc (Dunedin Fla.) Model LS 450 light source and S2000 spectrometer coupled with a ZR600-UV-F-SF probe. As a prototype example, we monitored changes in fluorescence associated with changes of pH. For this application we used fluorescein as the active molecule and wavelengths of 488 nanometers for excitation and measured emission at 520 nanometers. Independent pH measurements used an Orion 290A microprobe.

The general procedure outlined in Example 2 for covalent coupling of amines to endogenous carboxyl groups was used to attach fluorescein-dextran-lysine (FDL; Molecular Probes Inc, Corvallis Oreg.; product D-1820) to discs of RoTrac® Capillary-pore membrane with pores 0.1 micrometer in diameter. The FDL material, with fluorescein spaced on the dextran “backbone” at distances to minimize fluorescence quenching, allowed direct labeling of the membrane without generation of spectroscopic artifacts. Such configured capillary pore (fluorescent) membrane was immobilized in front of the light beam from the fiber optic probe to allow facile interrogation of the system. In preliminary studies (data not presented), changing concentration of FDL in the solution used to configure the capillary-pore membrane from 0.01 to 1 milligrams per milliliter resulted in proportional changes in membrane associated fluorescence.

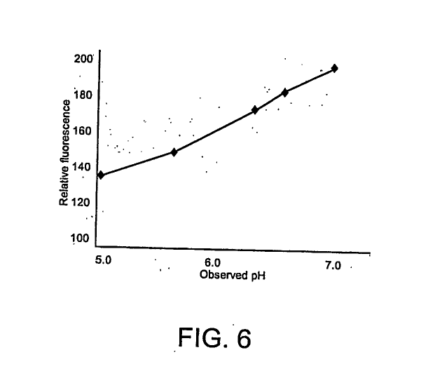

One use of FDL as the active molecule is measurement of pH. Discs of configured capillary pore membrane were immersed in buffers, agitated slightly, and allowed to sit for 20-60 min to allow diffusion into the pores. The intensity of emitted light was measured, and compared with the independently measured pH of each buffer. The pH of the bathing solution was adjusted, and the sample treated as above. As shown in FIG. 6, the response over the range of pH 5.0 to 6.8 illustrates use of this optical bench. In this example the signal-to-noise ratio, relative to a membrane without FDL, was found to be >100 to 1 (data not presented).

### EXAMPLE 4

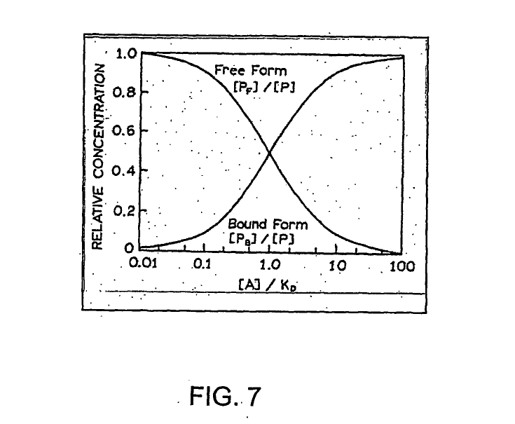

In Example 4, we estimated the theoretical limits of systems described in Example 3, and these estimates also apply to systems used in other examples. The core assumption was that the molecule “A” exists in free, A_{f}, and bound, A_{b}, states, with total number of binding sites “P” on the active molecules of the configured separation membrane (or any other competing molecule) present at a much lower concentration than that of the molecule. At a binding stoichiometry of 1:1, Equation 1 defines the dissociation reaction as AP_{b}_{_{—}}A+P_{f}, and the resultant dissociation constant (Kd) is defined in Equation 2 as Kd=(P_{f}(A)/(AP_{b}). Since the total concentration of binding protein is described by Equation 3, (P)=(P_{f})+(P_{b}), the relative concentrations of the free and bound forms of the binding protein. FIG. 7 presents a graphic illustration of the relationship between these variables. It shows molecule A could be bound accurately (providing a useful and reproducible plot of the change in fluorescence, etc) over an approximately 100-fold range using an appropriately configured separation membrane, assuming that Kd is between 0.1 and 10. And analogous approach can be used for features for extraction of molecules from a solution (Example 8) and optical analysis (Example 3).

The diversity of active molecules that could serve as an optical bench will allow use of appropriately configured separation membranes for many different analytes, as introduced in the Background section. Although outside the scope of this invention, it is obvious to one skilled in the art that a device incorporating a configured separation membrane designed as an optical bench could be a construct facilitating: (a) use as a static monitor, where the configured separation membrane is positioned to receive analyte moved therein by diffusion, possibly aided by mixing to insure equilibrium; (b) use in a flow-through monitor, where fluid to be analyzed flows through the configured separation membrane; (c) use in an assembly of multiple configured separation membranes, in a “stack” arrangement, allowing pulsed or intermittent analysis of several different analytes in one system (possibly <0.1 mm thick) through use of spectroscopic properties specific for analytes of interest; (d) use of one or more appropriately configured separation membrane(s) in a fluorescence (source and detector signals gathered in one physical device), absorbance (source and detector separated), or combination mode; and (e) analytical use of a configured separation membrane in which the active molecule emits a signal detectable by methods other than fluorescence or absorbance approaches.

### EXAMPLE 5

Given the number of active molecules that might be positioned within 1 cm² of a properly configured separation membrane, use in a process stream to expose molecules to catalyst to transform molecules during transmembrane passage of a solution is logical. Hicke et al. (1999) provided a convincing demonstration of use of an Oxyphen AG capillary-pore network as a bioreactor for synthesis of polysaccharide from fructose substrate via immobilized frutosyltransferase. In their process, Hicke et al. (1999) prepared the capillary pore membrane so that the desired functional residue was available, using a photoinitiated copolymerization of 2-aminoethyl methacrylate with the membrane, followed by extensive washing of the material, and immobilization of the enzyme with glutaraldehyde. All of their preliminary steps can be avoided through simple use of the endogenous carboxyl groups, first disclosed herein, and common carbodiimide chemistry as used in Example 1. This allows use of an enzyme as an active molecule and simple configuration of the capillary-pore membrane therewith. Other forms of catalyst could be used as active molecules, and any process where a vectorial movement of input materials through a catalytic center and exit products have value would be a potential use for an appropriately configured separation membrane.

### EXAMPLE 6

A configured separation membrane positioning many environmentally sensitive “gates” that could open or close, and especially “reversible gates”, within the transmembrane passageways would have many uses. Active molecules forming such gates could be designed to open or close in response to a variety of stimuli impacting the active molecules via solutions entering the pores, acting on the membrane per se, or both. Such membranes could automatically maintain a predefined differential or gradient across the configured separation membrane for a preselected stimulus molecule.

Polymers that change physical features in response to environmental changes are useful to selective and reversible occlusion of the pores to limit passage of molecules/particles through the capillary-pore membrane. Polymers that respond to alterations of local environment, such as temperature, chemical potential, electrochemical potential, pressure, or electromagnetic radiation are suitable for this use. As one of many possible examples, an α-helix of a peptide occupies a linear distance of ˜0.54 nanometers for every 3.6 amino acid residues, but when allowed (or driven) to its alternative form of a random coil the linear distance increases 2-4 fold. Thus, a peptide containing ˜200 amino acids (MW ca 24,000 daltons) would occupy a linear distance of 30 nanometers (0.03 micrometers) in its compact form and 60-120 nanometers (0.06-0.12 micrometers) in its expanded form. For a capillary-pore membrane with pores of 0.1 micrometer diameter, such a peptide could extend a considerable distance across the pore. Assuming that such active molecules were attached at a concentration of ˜4 nanomoles per cm² (Table 2), millions of peptides (˜10% that achieved for histidine in Example 2, and used in calculations above) could be placed within the pore (assuming entry and covalent linkage with endogenous carboxyl groups while in the compact form).

Alternative approaches to provide a “gate” polymer, responsive to changes in its chemical environment, are envisioned. Examples are provided for protein polymers, but other chemical compositions are possible and this invention includes any polymer that changes configuration and the “occupied space” as an active molecule.

First, consider examples where the gate polymer is temporarily “locked open” in its folded conformation. That locking can be accomplished by intrachain bonding between repeating units of the polymer. As examples, critically spaced cysteines can be converted to disulfide, intrachain, cystines. When in this state, the polymer will be hindered from assuming its extended, unfolded form. However, when a reducing agent accumulates in the medium, surrounding the gate, disulfide bonds will be reduced and broken, and the polymer can assume its extended or “closed gate” form. In a second example, critically spaced hydroxyl groups (e.g., serine or threonine) can connected by intrachain boronate esters (by reaction of boric acid with hydroxyl functions) to form a temporarily “locked open” polymer. When exposed to polyols (e.g., glucose, glycerol) an exchange reaction will take place, passing the boronate to the exogenous polyol, and allowing the polymer to assume its extended or “closed gate” form.

The converse also is possible. Consider examples where the polymer is temporarily “locked closed” in its extended form, bridging the pore. This could be accomplished in a multistage reaction. Using a protein as an example, consider an N terminal function with a free amine, a chemically responsive group (e.g., adjacent hydroxyl groups) within the chain, and a C terminal possessing a protected sulfhydryl function. In Step 1, endogenous carboxyl groups of the capillary-pore membrane are activated (as in Example 2). Then, the active molecule is positioned within transmembrane passageways, and the free amine of the active molecule is allowed to bond activated endogenous carboxyl groups via its N terminal. Reaction conditions are chosen so that only a portion of the carboxyl groups are consumed. The C terminal is free, and can extend across the pore to close contact with the opposite wall. In Step 2, the protecting function is removed to expose the C terminal sulfhydryl. In Step 3, the remaining carboxyl groups on the pore surface are activated, and allowed to react with the C terminal to form a thiol ester. This completes configuration of the gated-pore membrane, and in such format the contractile protein is “locked closed” across the pore by covalent bonds to its C and N terminals. The spacing and density of such “locked closed” active molecules along the wall of each transmembrane passageway make the configured separation membrane effectively impermeable. When conditions suitable for cleaving the chain are encountered, the polymer is cleaved and the contractile protein assumes a compact form and flow through the transmembrane passageways can occur.

### EXAMPLE 7

We considered that selectively addition of either hydrophilic or hydrophobic molecules to a surrounding solution could be an important use of an appropriately configured separation membrane. One approach for accomplishment of this use was to use a “host-guest” combination as the active molecule. By explanation, the “host” would be a molecule that could be covalently bound to endogenous carboxyl residues while it was pre-loaded with a “guest” molecule, not covalently bound to the host or the membrane, that would be released from the host when it was exposed to appropriate conditions. Many examples could be envisioned by one skilled in the art. To illustrate this approach, we discuss cyclodextrins (see Background section) because they offer a unique structure with a polar outer surface and a hydrophobic inner core. Chemistry of the outer wall of a cyclodextrin should allow covalently linked to endogenous carboxyl groups while the inner core contains lipid-like molecules. Indeed, cyclodextrins have been used widely to hold hydrophobic molecules in a pseudo water-soluble form. This allows a properly configured separation membrane to bring individual molecules (not films or aggregates) of hydrophobic molecules in direct contact with an aqueous solution passing through the transmembrane passageways. When solution conditions change, such as temperature, presence of other solutes, physical features (e.g., light action on a photoswitchable cyclodextrin) the equilibrium constraints holding the guest within the host alter the host-guest relationship and release guest to the environment. Preferred uses of capillary-pore membranes configured in this manner would include release of pharmaceuticals, biological regulators, analytical signaling agents, etc. The features of FIG. 7 also describe the release of guest molecules from a host-guest complex.

### EXAMPLE 8

We evaluated the concept of using an “affinity-ligand” as the active molecule, to selectively remove a molecule from a solution on the basis of high and specific affinity of active molecule for the molecule(s) to be removed. An affinity-ligand ideally is positioned to provide selective interaction with solutes or ions in a solution passing through an appropriately configured separation membrane. Such uses can be achieved through use of any active molecule with high affinity for a specific solute(s), but frequently would be accomplished by use of antibodies, receptor proteins, or one member of a highly evolved system (e.g., biotin-avidin) as the active molecule. Hence, we used avidin as the affinity-ligand.

For the study of Example 8, we used the method described in Example 2 to configure (covalently bind) a capillary-pore membrane with avidin (0.01, 0.05 or 0.1 milligram/mL solution) to prepare membrane discs with three different amounts of avidin bound within the transmembrane passageways. We assumed that, within this range, increasing numbers of avidin molecules would be bound, and would be able to extract increasing amounts of a biotin-containing compound from solution.

These configured separation membrane discs then were exposed to biotin-fluorescein-dextran-lysine (BFDL; Molecular Probes Inc, Corvallis Oreg.; product D-7178) at a concentration of 0.167 milligrams/mL. Fluorescence from BFDL extracted by the affinity-ligand was measured as in Example 3 with results (concentration of avidin used to treat membrane; mgm/mL: fluorescence units observed after treatment with constant amount of BFDL and instrument settings) of: 0.01:145,000; 0.05:350,000; 0.1:500,000. Such results are consistent with increased capacity to extract material from solution by increased amounts of affinity-ligand on the membrane.

The alternative experiment, use of identical discs of capillary-pore configured with the same concentration of avidin and testing extraction from a range of concentrations of BFDL in solution, was not conducted. The high affinity of d-biotin for avidin (Ka˜2×10¹³ M⁻¹) precludes this experiment. Because BFDL is quantitatively extracted from the solution at the lowest concentrations detectable, measurement of a dose response would be impossible.

### DESCRIPTION OF THE DRAWINGS

FIG. 1 presents a micrograph of the Mitex membrane system, as sold by Millipore Inc and used by Ito et al. Taken from Millipore promotional materials. Note large range in pore sizes.

FIG. 2 presents micrographs of capillary-pore membrane, as sold by Oxyphen AG. Taken from Oxyphen AG promotional materials. Note the isoporous (homogeneous) nature of the capillary network.

FIG. 3 presents photographic evidence of association of Neutral Red dye with various capillary-pore membrane materials. In the top row, “polyester solid” represents the base membrane materials used in capillary-pore manufacture before treatment to make the pores (this optically clear material did not take up dye, and is no visible in photograph), while the same material after treatment to form 10 micrometer pores is lightly stained by the dye. Increasing amounts of dye are associated with those membranes with the smallest pore materials, with a higher intensity when the membranes are mounted with the “smooth side” (bearing the capillary-pore network) up, rows 2-6.

FIG. 4 presents photographic evidence for the loss of Neutral Red dye binding capacity after treatment (for 1-4 hr) with a carboxyl activating reagent and high concentrations of ammonium chloride.

FIG. 5 presents a schematic representation of the process described in FIG. 4. Panel A illustrates the base feature of the capillary-pore membrane (as in FIG. 2). Panel B illustrates the presence of carboxyl groups within the pore, as suggested by data of FIG. 3 and Table 1. Panel B illustrates the conversion of the carboxyl groups to amides, as suggested by data of FIG. 4. Carboxyl groups can be converted to many other derivatives (alcohols, anhydrides, etc.) that are useful for covalent attachments by methods known to those skilled-in-the-art.

FIG. 6 presents results of a test of immobilized fluoroscein-dextran-lysine to assess changes in pH of the solution bathing the capillary-pore membrane.

FIG. 7 illustrates the relationship between relative concentration of free and bound forms of binding protein, molecule concentration and dissociation constant. Taken from Lakowicz (1999).

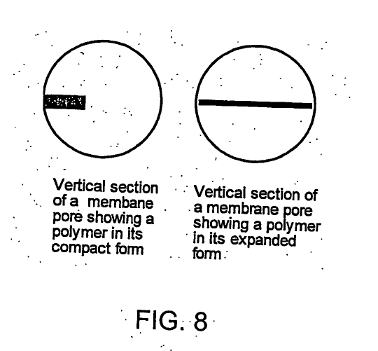

FIG. 8 presents a schematic representation of a covalently attached polymer capable of existing in a compact form and expanded (right) forms.

### LITERATURE CITED

U.S. Pat. No. 5,026,342
U.S. Pat. No. 5,261,870
U.S. Pat. No. 6,399,363
Anderson J L (1971) Characterization of mass and charge transport: development of a model membrane. PhD Thesis, U Illinois.
Awaji T, Hirasawa A, Shirakawa H, Tsujimoto G and Miyazaki S (2001) Novel green fluorescent protein-based ratiometric indicators for monitoring pH in defined intracellular microdomains. Biochem Biophys Resh Commun 289: 457-462.
Badugu R, Lakowicz J R and Geddes C D (2003) A glucose sensing contact lens: A non-invasive technique for continuous physiological glucose monitoring. J Fluorescence 13: 371-374.
Baird G S, Zacharias D A and Tsien R Y (1999) Circular permutation and receptor insertion within green fluorescent proteins. Proc Natl Acad Sci USA 96: 11241-11246.
Becker M, Provart N, Lehmann L Ultricht M and Hicke H-G (2002) Polymerization of glucans by enzymatically active membranes. Biotechnol Prog 18: 964-968.
Billinton N and Knight A W (2001) Seeing the wood through the trees: A review of techniques for distinguishing green fluorescent protein from endogenous autofluorescence. Anal Biochem 291: 175-197.
Borcherding H, Hicke H G, Jorcke D and Ulbrecht M (2003) Affinity membranes as a tool for life sciences applications. Ann NY Acad Sci. 984: 470-479.
Chiesa A, Rapizzi E, Tosello V, Pinton P, de Virgilio M, Fogarty K E and Rizzuto R (2001) Recombinant aequorin and green fluorescennt protein as valuable tools in the study of cell engineering. Biochem J 355: 1-12.
D'Souza V T and Lipkowitz K B (1998) “Cyclodextrins” Chemical Reviews 98: 1741-2074.
Elsliger M A, Wachter R M, Hanson G T, Kallio K and Remington S J (1999) Structural and spectral response of green fluorescent protein variants to changes in pH. Biochemistry 38: 5296-5301.
Fleischer R L and Price P B (1963a) Charged particle tracks in glass. J Appl Phys 34: 2903-2904.
Fleischer R L and Price P B (1963b) Tracks of charged particles in high polymers. Science 40: 1221-1222.
Fleischer R L, Price P B and Symes, E M (1964) Novel filter for biological materials. Science 143: 249-250.
Griesbeck O, Baird G S, Campbell R E, Zacharais D A and Tsien R Y (2001) Reducing the environmental sensitivity of yellow fluorescent protein. Mechanism and applications. J Biol Chem 276: 29188-29194.
Hermanson G T (1996) “Bioconjugate Techniques” Academic Press, San Diego.
Hicke H-G, Ulbricht M, Becker M, Radosta S and Heyer A G (1999) Novel enzyme-membrane reactor for polysaccharide synthesis. J Membrane Sci 161: 239-245.
Holtzer A (1994) Application of old and new values of α-helix propensities to the helix-coil transition of poly(L-glutamic acid). J Am. Chem. Soc 116: 10837-10838.
Hughes-Despointes, BMP, Scholtz J M and Baldwin R L (1993) Helical peptides with three pairs of Asp-Arg and Glu-Arg residues in different orientations and spacings. Polymer Sci 2:80-85.
Ito Y, Kotera S, Inaba M, Kono K and Imanishi Y. (1990) Control of pore size of polycarbonate membrane with straight pores by poly(acrylic acid) grafts. Polymer 31: 2157-2161.
Ito Y, Inaba M, Chung D-J and Imanishi Y (1992) Control of water permeation by pH and ionic strength through a porous membrane having poly(carboxylic acid) surface-grafted. Macromolecules 25: 7313-7316.
Ito Y, Ochiai Y, Park Y S and Imanishi Y (1997) pH Sensitive gating by conformational change of a polypeptide brush grafted onto a porous polymer membrane. J Am Chem Soc 119:1619-1623.
Ito Y, Suzuki M and Husimi Y (1999) A novel mutant of green fluorescent protein with enhanced sensitivity for microanalysis at 488 nm excitation. Biochem Biophys Res Commun 264: 556-560.
Janicke, L (1974) A rapid micromethod for the determination of nitrogen and phosphate in biological material Anal Biochem 61: 623-627.
Kesting R E (1971) Synthetic Polymeric Membranes, McGraw-Hill New York.
Kinoshita T, Iwata T, Takizawa A and Tsujita Y (1983) Ionic salt permeabilities and membrane potentials of asymmetric polypeptide membrane. Colloid & Polymer Sci 261: 933-937.
Kinoshita T, Kakiuchi Takizawa A, Tsujita Y, Oya M, Iizuka Y and Iwatsuki M (1994) Solute permeability enhancement at a specific pH by an amphiphilic copolypeptide membrane. Macromolecues 27: 1389-1394.
Kneen M, Farinas J, Li Y and Verkman A S (1998) Green fluorescent protein as a noninvasive intracellular pH indicator. Biophys J 74: 1591-1599.
Lakowicz J R (1999) “Principles of Fluorescence Spectroscopy; Second Edition” Klywer Academic/Plenum Publishers, New York
Liu Y, Chen Y, Li L, Huang G, You C-C, Zhang H-Y, Wada T and Inoue Y (2001a) Cooperative multiple recognition by novel calix(4)arene-tethered β-cyclodextrin and calix(4)arene-bridged bis(β-yclodextrin) J Org Chem 66: 7209-7215.
Liu Y, You C-C and Li B (2001b) Synthesis and molecular recognition of novel oligo(ethylenediamino) bridged bis(β-cyclodextrin)s and their copper (II) complexes: Enhanced molecular binding ability and selectivity by multiple recognition. Chem Eur J 7: 1281-1288.
Llopis J, McCaffery J M, Miyawaki A, Farquhar M G and Tsien R Y (1998) Measurement of cytosolic, mitochondrial, and Golgi pH in single living cells with green fluorescent proteins. Proc Natl Acad Sci USA 95: 6803-6808.
Lovrien R and Linn T (1967) Mutual distortion mechanisms in macromolecular catalysis. Biochemistry 6: 2281-2293.
Lovrien R, Pesheck P and Tisel W (1974) Protein and hydrogen ion control of photochromism in aminoazobenzene compounds. J Am Chem Soc 96: 244-248.
Luck H B (1983) On the use of polyethyleneterephtalate as a solid state nuclear track detector: Kinetics and mechanism of particle track etching. Nuclear Instruments and Methods 213: 507-511.
Maeda M, Kimura M, Hareyama Y and Inoue, S (1984) pH-dependent ion transport across polymer membrane. pH-induced reversible conformational change of transmembrane poly(L-aspartic acid) domain in polymer membrane. J Am Chem Soc 106: 250-251.
Margolin W (2000) Green fluorescent protein as a reporter for macromolecular localization in bacterial cells. Methods 20-62-72.
Miyawaki A, Llopis J, Heim R, McCaffery J M, Adams J A, Ikura M and Tsien R Y (1997) Fluorescent indicators for Ca2+ based on green fluorescent proteins and calmodulin. Nature 388: 882-887.
Mulder A, Jukovic A, Lucas L N, van Esch J, Feringa B L, Huskens J and Reinhoudt D N (2002) A dithienylethene-tethered β-cyclodextrin dimer as a photoswitchable host. Chem Communications 21: 2734-2735.
Nagai T, Sawano A, Park E S and Miyawaki A (2001) Circularly permuted green fluorescent proteins engineered to sense Ca2+. Proc Natl Acad Sci USA 98: 197-202.
Nagai T, Ibata K, Park E S, Kubota M, Mikoshiba K and Miyawaki A (2002) A variant of yellow fluorescent protein with fast and efficient maturation for cell-biological applications. Nature Biotech 20: 87-90.
Nifosi R and Tozzini V (2003) Molecular dynamics simulations of enhanced green fluorescent proteins: effects of F64L, S65L and T203Y mutations on the ground-state proton equilibria. Proteins 51: 378-389.
Petzny W J and Quinn J A (1969) Calibrated membranes with coated pore walls. Science 166: 751-752.
Price P B, and Walker R M (1962a) Chemical etching of charged-particle tracks in solids. J Appl Phys 33: 3407-3412.
Price P B and Walker R M (1962b) A new track detector for heavy particle studies. Phys Letters 3: 113-115.
Quinn J A, Anderson J L, Ho W S and Petzny W J (1972) Model pores of molecular dimension. The preparation and characterization of track-etched membranes. Biophys J 12: 990-1007.
Rekharsky M V and Inoue Y (2002) Complexation and chiral recognition thermodynamics of 6-amino-6-deoxy-β-cyclodextrin with anionic, cationic, and neutral chiral guests: Counterbalance between van der Walls and coulombic interactions. J Am Chem Soc 124: 813-826.
Riedel C and Spohr R (1980) Transmission properties of nuclear track filters. J Membrane Sci 7: 225-234.
Robey R B, Ruiz O, Santos A V, Ma J, Kear F, Wang L J, Li C J, Bernardo A A and Arruda J A (1998) pH-dependent fluorescence of a heterologously expressed Aequorea green fluorescent protein mutant: in situ spectral characteristics and applicability to intracellular pH estimation. Biochemistry 37: 894-9901.
Staros J V, Wright R W and Swingle D M (1986) Enhancement by N-hydroxysulfosuccinimide of water-soluble carbodiimide-mediated coupling reactions. Anal Biochem 156:220-222.
Takahashi A, Zhang Y, Centonze E and Herman B (2001) Measurements of mitochondrial pH in situ. Biotechniques 30: 804-808.
Speck E J, Gong, Y and Kallenbach, N R (1995) Intermolecular interactions in α helical oligo- and poly(L-glutamic acid) at acidic pH. J Am Chem Soc 117: 10773-10774.
Staros J V, Wright R W and Swingle D M (1986) Enhancement by N-hydroxysulfosuccinimide of water-soluble carbodiimide-mediated coupling reactions. Anal Biochem 156: 220-222.
Szejtli J and Osa T (1996) Cyclodextrins, Vol 3 of “Comprehensive Supramolecular Chemistry” Pergamon Press, New York.
Uekama K and Otagiri M (1987) CRC Critical Reviews in Drug Carrier Systems 3: 1-40.
Urry D W (1997) Physical chemistry of biological free energy transduction as demonstrated by elastic protein-based polymers. J Phys Chem 101B:11007-11028.
Yi K and Schultz J S (2003) Genetic engineering of an allosterically based glucose indicator protein for continuous glucose monitoring by fluorescence resonance energy transfer. Anal Chem 75: 3451-3459.

1. A method for altering the properties of a capillary-pore membrane by linking at least one compound to said membrane via an endogenous carboxyl group inherent in said membrane.

2. The method according to claim 1, wherein said alteration of said capillary-pore membrane is by attachment of one or more molecules, particles, units of matter, or combination thereof within one or more of said transmembrane passageways via covalent linkage with said endogenous carboxyl groups using any chemical procedure, thereby forming a configured separation membrane.

3. The method according to claim 1, wherein said endogenous carboxyl groups are modified using any chemical procedure prior to covalent attachment of one or more of molecules, particles, units of matter, or combination thereof within one or more of said transmembrane passageways using any chemical procedure, thereby forming a configured separation membrane.

4. The method according to claim 2 wherein carbodiimide reaction is used to accomplish said alteration of said membrane by linkage of a molecule, particle, or unit of matter containing an amine group with said endogenous carboxyl groups.

5. The method according to claim 2 wherein carbodiimide reaction is used to accomplish said alteration of said membrane by linkage of a molecule, particle, or unit of matter containing a thiol group with said endogenous carboxyl groups.

6. The method according to claim 2 wherein the endogenous carboxyl groups are reacted to form anhydrides.

7. A method of use of a capillary-pore membrane configured as in claim 2, to assess and/or quantitatively describe changes in a solution by detection of changes in one or more physical or chemical properties of one or more of said molecules, particles, units of matter, or combination thereof positioned within one or more transmembrane passageways.

8. A method of use of a capillary-pore membrane configured as in claim 3, to assess and/or quantitatively describe changes in a solution by detection of changes in one or more physical or chemical properties of one or more of said molecules, particles, units of matter, or combination thereof positioned within one or more transmembrane passageways.

9. A method of use of a capillary-pore membrane configured as in claim 2, to assess and/or quantitatively describe changes in a solution by detection of changes in one or more physical or chemical properties of one or more fluorescent molecules positioned within one or more transmembrane passageways.

10. A method of use of a capillary-pore membrane configured as in claim 3, to assess and/or quantitatively describe changes in a solution by detection of changes in one or more physical or chemical properties of one or more fluorescent molecules positioned within one or more transmembrane passageways.

11. A method of use of a capillary-pore membrane configured as in claim 2, to assess and/or quantitatively describe changes in a solution on the basis of changes in absorbance or physical spectrum of one or more molecules positioned within one or more transmembrane passageways.

12. A method of use of a capillary-pore membrane configured as in claim 3, to assess and/or quantitatively describe changes in a solution on the basis of changes in absorbance or physical spectrum of one or more molecules positioned within one or more transmembrane passageways.

13. A method of use of a capillary-pore membrane configured as in claim 2, to modify a solution via catylatic, enzymatic, or other actions performed by one or more molecules, particles, units of matter, or combination thereof positioned within one or more transmembrane passageways.

14. A method of use of a capillary-pore membrane configured as in claim 3, to modify a solution via catylatic, enzymatic, or other actions performed by one or more molecules, particles, units of matter, or combination thereof positioned within one or more transmembrane passageways.

15. A method of use of a capillary-pore membrane configured as in claim 2, to control flow rate of fluid through one or more transmembrane passageways, or to initiate, terminate, or in a reversible manner initiate and terminate flow of fluid through one or more transmembrane passageways.

16. A method of use of a capillary-pore membrane configured as in claim 3, to control flow rate of fluid through one or more transmembrane passageways, or to initiate, terminate, or in a reversible manner initiate and terminate flow of fluid through one or more transmembrane passageways.

17. A method of use of a capillary-pore membrane configured as in claim 2, to add a solute, ion, or other matter to a solution via release from one or more molecules, particles, units of matter, or combination thereof positioned within one or more transmembrane passageways.

18. A method of use of a capillary-pore membrane configured as in claim 3, to add a solute, ion, or other matter to a solution via release from one or more molecules, particles, units of matter, or combination thereof positioned within one or more transmembrane passageways.

19. A method of use of a capillary-pore membrane configured as in claim 2, to selectively remove a solute, ion, or other matter from a solution via binding or containment by one or more molecules, particles, units of matter, or combination thereof positioned within one or more transmembrane passageways.

20. A method of use of a capillary-pore membrane configured as in claim 3, to selectively remove a solute, ion, or other matter from a solution via binding or containment by one or more molecules, particles, units of matter, or combination thereof positioned within one or more transmembrane passageways.

21. A method of use of a capillary-pore membrane configured as in claim 2, to selectively monitor changes in the environment near or within said transmembrane passageways including chemical potential, electrochemical potential, electromagnetic radiation, light, osmotic force, pH, or temperature.

22. A method of use of a capillary-pore membrane configured as in claim 3, to selectively monitor changes in the environment near or within said transmembrane passageways including chemical potential, electrochemical potential, electromagnetic radiation, light, osmotic force, pH, or temperature.

23. A method of use of a capillary-pore membrane configured as in claim 2, to provide remedial action, to counter environmental changes detected by one or more molecules, particles, units of matter, or combination thereof positioned within said transmembrane passageways, via release of one or more appropriate materials from within or through the same or other transmembrane passageways.

24. A method of use of a capillary-pore membrane configured as in claim 3, to provide remedial action, to counter environmental changes detected by one or more molecules, particles, units of matter, or combination thereof positioned within said transmembrane passageways, via release of one or more appropriate materials from within or through the same or other transmembrane passageways.
